(19) United States
(12) Patent Application Publication
(10) Pub. No.: US 2010/0252049 A1
(43) Pub. Date: Oct. 7, 2010

(54) Endotracheal tube securing device

(76) Inventor: Louis G. Kost, Owensboro, KY (US)

Correspondence Address: David W. Carrithers;Carrithers Law Office, PLLC, The 1000 Building, 6200 Dutchman's Lane, Suite 206, Louisville, KY, 40205, US

(21) Appl. No.: 12/384,247

(22) Filed: Apr. 2, 2009

(51) Int. Cl. A61M 16/04

(52) U.S. Cl. 128/207.17

(57) ABSTRACT

An endotracheal securing device includes a main tube and a tie extending through the main tube. The tie projects out through a central orifice in the main tube and is tightened around the endotracheal tube. The ends of the tie are then tied behind the user's head to secure the tie and endotracheal tube in place between the user's nose and mouth.

### TECHNICAL FIELD

The present invention relates to a mechanism for securing and supporting an oral or nasal endotracheal tube on a patient.

### DESCRIPTION OF THE RELATED ART

U.S. Pat. No. 4,437,463 by Ackerman for “SECURING DEVICE FOR TUBE INSERTABLE IN BODY CAVITY” issued May 20, 1984 teaches forming a noose in thin-walled elastomeric tubing by means of the passage of the ends of the tubing through a ring of a thick walled tubular section. The noose is drawn tight around the device to be held (e.g. an endotracheal tube). One end of the tube is fastened on a clamp or hood and the other end is held fast by the clamp or hook.

U.S. Pat. No. 5,042,477 by Lewis for “MEDICAL TUBE HOLDER” issued Aug. 27, 1991 teaches a tie within a small length of tube. The tie within the tube is pulled through a slit in the center of the tube forming a loop. An endotracheal tube is fed through the loop. The free ends of the tie are pulled to tighten the loop around the endotracheal tube and are tied behind or to the side of the user's head to secure the tube in the user's mouth.

### BACKGROUND OF THE INVENTION

Prior devices have been too bulky, unstable, difficult to work with and not very versatile. Previous devices have also used adhesive tapes and or regular tape that has proven very cumbersome and not easily used in an emergency situation. Adhesive devices and tape also become loose when wet and soiled and tend to break down the patient's skin.

Ackerman uses tubing to hold the endotracheal tube. It is cumbersome work with and to slide the tubes into place against one another. In addition, the hook or clamp would be uncomfortable behind or beside the user's head and the tubing could stretch to allow the endotracheal tube to move and possibly pull out of the user's mouth.

Lewis teaches a tie which protrudes through a slit in the middle of a length of tube to form a loop and is then tied around the user's head. A double loop resembling a figure ‘8’ is then used to hold the endotracheal tube. This double loop will not hold the tube firmly enough and will loosen in time.

### SUMMARY OF THE INVENTION

The present invention provides a user friendly device for quickly and comfortably securing an endotracheal tube to a patient's nose or mouth. It also can be removed, replaced, or repositioned easily.

An endotracheal tube securing device presented herein comprises a main tube, including a tubular wall defining an interior, an exterior, and first and second ends. The tubular wall also has an aperture extending through the tubular wall half way down the length of the tube. A tie extends along the interior of the main tube and out the first and second ends, wherein a portion of the tie protrudes through the aperture and forms a clove hitch knot outside of the main tube. Means for tightening the tie around a patient's head are included.

It is an object of this invention to provide an endotracheal tube securing device which is simple, inexpensive and easy to manufacture.

It is an object of this invention to provide an endotracheal tube securing device which holds the tube firmly and dependably in place.

It is an object of this invention to provide an endotracheal tube securing device which is comfortable to the user and easy to install.

### BRIEF DESCRIPTION OF THE DRAWINGS

A better understanding of the present invention will be had upon reference to the following description in conjunction with the accompanying drawings in which like numerals refer to like parts throughout the views wherein:

FIG. 1 is a plan view of one embodiment of an endotracheal tube securing device made in accordance with the present invention;

FIG. 2 is a view of the securing device of FIG. 1, in which a clove hitch knot has been formed on the tie of FIG. 1, and an endotracheal tube has been extended through the knot;

FIG. 3 is a view of the securing device of FIG. 2, in which the clove hitch knot has been formed and held in place with tape;

FIG. 4 is a view of the securing device of FIG. 3 with the clove hitch knot tightened around the endotracheal tube;

FIG. 5 is a view of another embodiment of an endotracheal tube securing device including a second tube and tie;

FIG. 6 is a view of the securing device of FIG. 2 as it is positioned on a patient's face;

FIG. 7 is a view of the securing device of FIG. 2, with the spacer tube removed and the device positioned on a patient's face; and

FIG. 8 is a view of the endotracheal tube securing device of FIG. 7, wherein the portion of the tie in contact with the patient is covered by a pad or tube to minimize irritation to the wearer.

### DESCRIPTION OF THE PREFERRED EMBODIMENTS

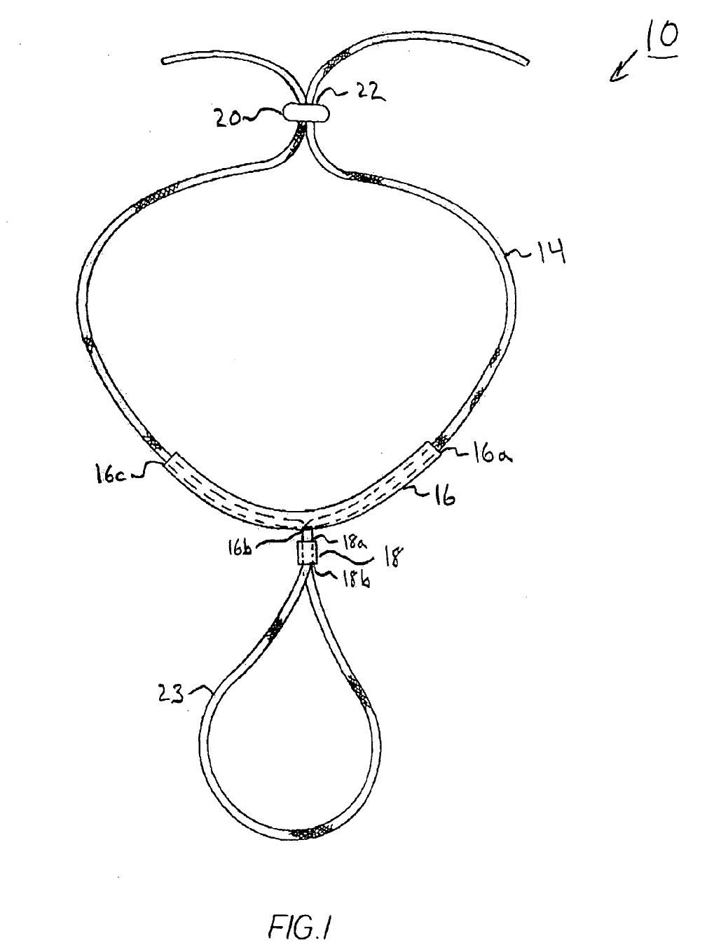

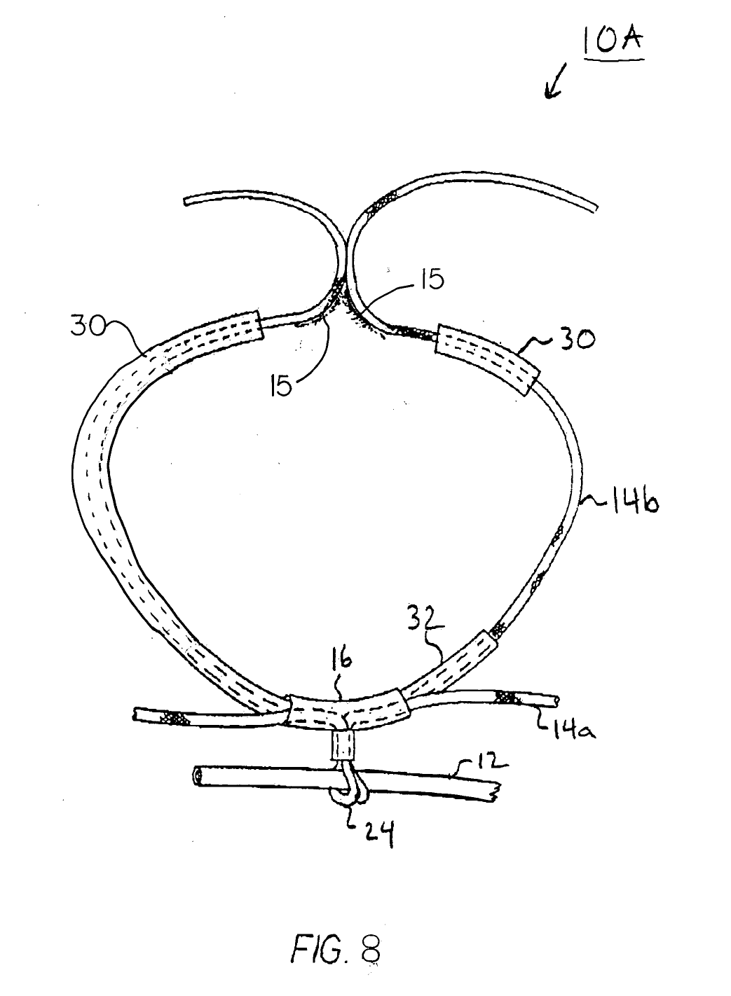

In accordance with the present invention, herein is presented an endotracheal tube securing device. FIGS. 1-4 show one embodiment of an endotracheal tube securing device 10 made in accordance with the present invention. Securing device 10 includes tie 14, main tube 16, spacer tube 18, and means for tightening tie 14 around the patient's head, which, in this case, is bead 20, which holds tie 14 in place by means of a friction fit between internal hole 22 in bead 20 and the outer surface of tie 14. Of course, other means for tightening tie 14 could be utilized, such as simply tying both ends of tie 14 together or utilizing hook and loop fasteners 15 as shown in FIG. 8.

The purpose of main tube 16 is to minimize contact between tie 14 and the patient's face and to allow tie 14 to be shifted relative to the patient's face without rubbing against the patient's skin. In this embodiment, tube 16 has a cylindrical cross-sectional shape, which provides for a limited contact surface between tube 16 and the patient's face, thereby diminishing the chance for skin breakdown. In this embodiment, tube 16 is made of a pliable, non-latex, plastic.

Tie 14 extends from a first end 16a of main tube 16, along the interior of main tube 16, out through central orifice 16b through the wall of main tube 16, through a first end 18a of spacer tube 18, along the interior of spacer tube 18, and out a second end 18b of spacer tube 18, where it forms loop 23. Tie 14 then returns back in through the second end 18b of spacer tube 18, along the interior of spacer tube 18, out the first end 18a of spacer tube 18, back through central orifice 16b of main tube 16, along the interior of main tube 16, and out through a second end 16c of main tube 16.

Spacer tube 18 provides an extra degree of comfort because tube 16 may rest comfortably under and against the user's nose instead of being held against the endotracheal tube by a tightened loop 23 and down over the user's upper lip in an uncomfortable position.

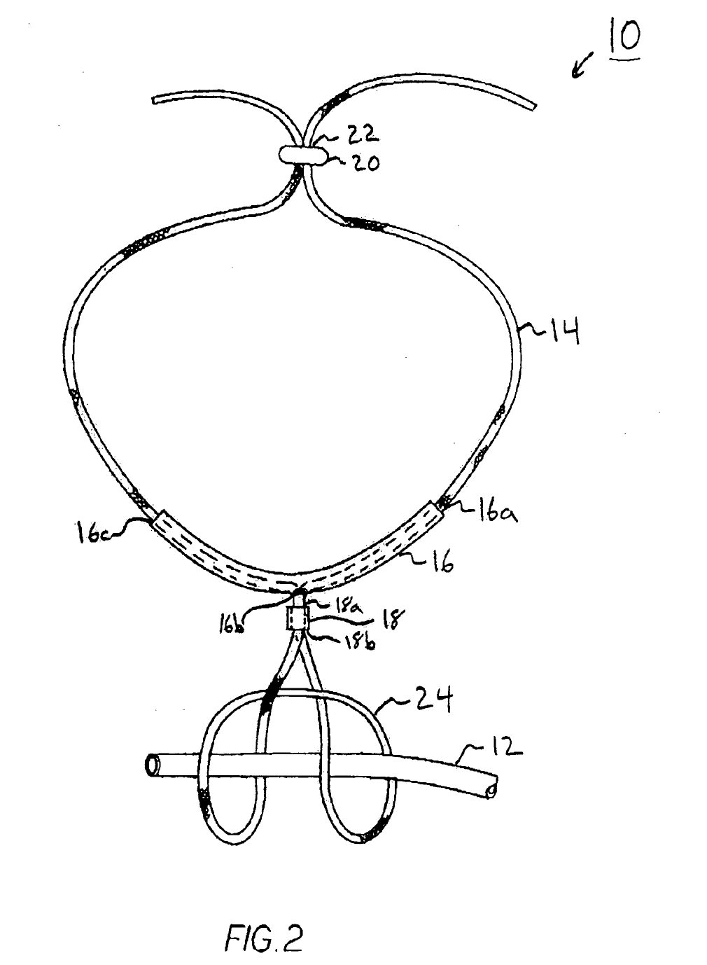

FIG. 2 shows the embodiment of FIG. 1, in which loop 23 has been formed into clove hitch knot 24, in order to firmly grip endotracheal tube 12 to reduce the chance of slippage or accidental removal of endotracheal tube 12. In this embodiment, tie 14 is made of a cloth that is stitched with a thick thread on both sides in order to improve the grip on endotracheal tube 12 when knot 24 is tightened around tube 12. In a prototype, radio-opaque tape, made of cotton and having a radio-opaque gripping thread, has been used.

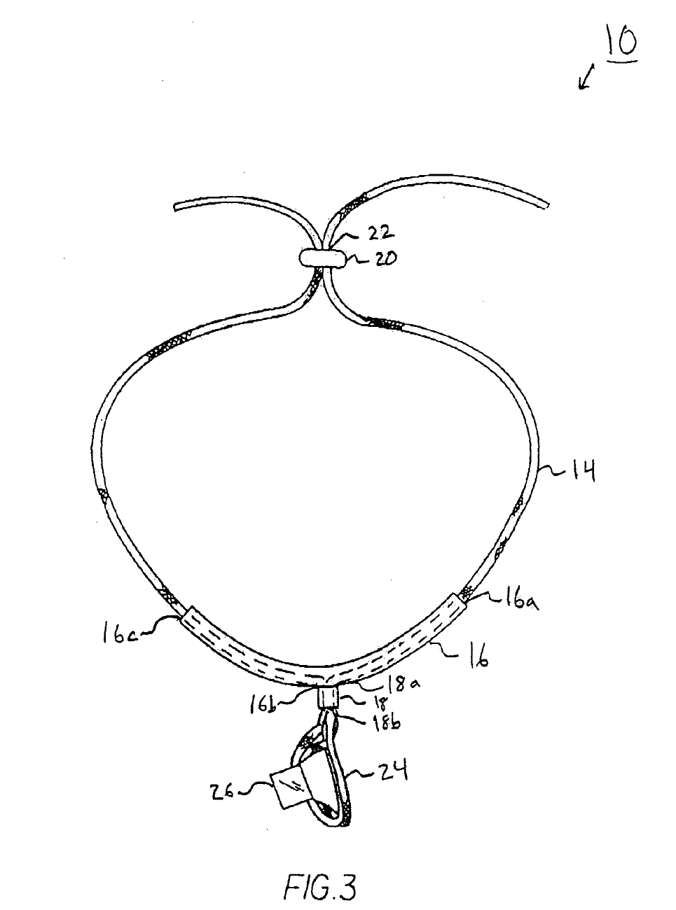

As shown in FIG. 3, clove hitch knot 24 has been pre-formed and held in position by a removable piece of paper tape 26. This arrangement ensures that the nurse or person responsible for securing the tracheal tube does not have to take the time to form the knot. Of course, other means besides paper tape, such as a twist tie or a simple piece of string tied around knot 24, may be used to hold knot 24 in place until it is tightened around the endotracheal tube.

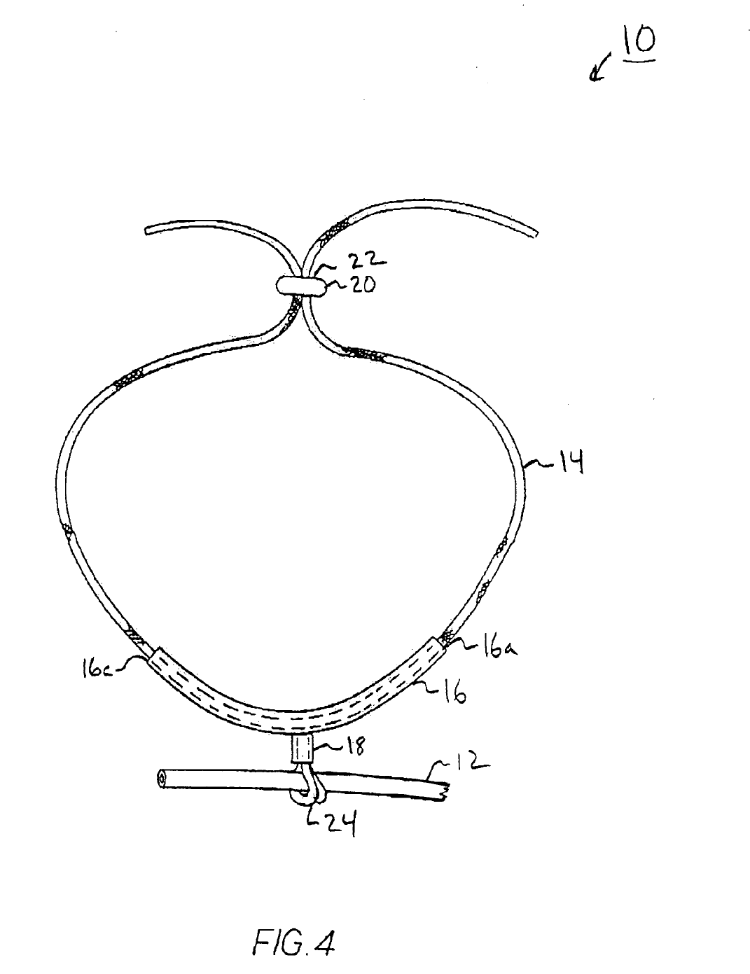

In the typical use of securing device 10, a nurse slips pre-tied knot 24 over the end of the endotracheal tube, and slides knot 24 down to the point along endotracheal tube 12 where it is to be secured. Then, he removes paper tape 26 that is holding knot 24, and pulls tie 14 so that knot 24 tightens around tube 12 until snug. Next, the nurse grasps the ends of tie 14 that lie beyond ends 16a, 16c of main tube 16 and pulls until main tube 16 touches spacer tube 18, and the tightened knot 24 abuts spacer tube 18. When knot 24 is securely tightened onto endotracheal tube 12, and main tube 16 is in the desired position, tie 14 is then pulled through bead 20 to tighten tie 14 onto the patient's head. FIG. 4 shows a view of securing device 10 when knot 24 is tightened snugly around endotracheal tube 12.

Sometimes it is desirable to reposition endotracheal tube 12 from one side of the mouth to the other to prevent skin break down to the patient's lip and mouth area. To reposition endotracheal tube 12, the nurse simply supports endotracheal tube 12 with his fingers and slides device 10 in the desired direction. The nurse does not have to tie knot 24 or remove securing device 10 from the patient's head.

When it is time to take tracheal tube 12 out of the trachea and out of the mouth, the nurse simply loosens tie 14 by sliding it through bead 20, deflates the cuff (not shown) of the tracheal tube, and then, using main tube 16 as a handle, grasps main tube 16 and tracheal tube 12 and pulls tracheal tube 12 out.

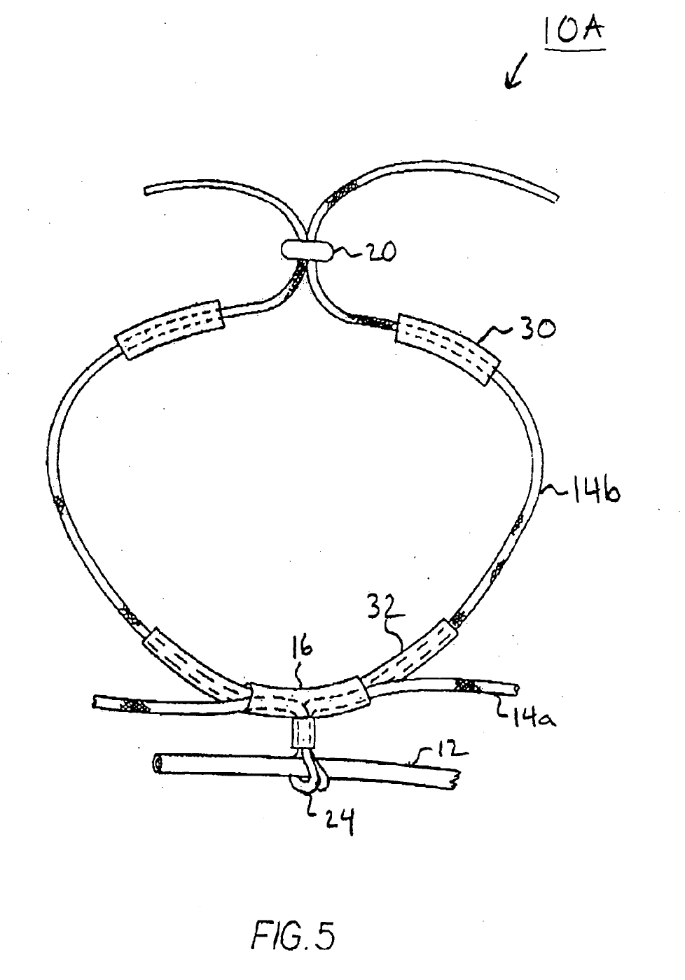

FIG. 5 shows another embodiment of an endotracheal tube securing device 10A in which there are two separate ties—a first tie 14a, which carries knot 24, and a second tie 14b, which secures main tube 16 to the patient's head. In this case, main tube 16 fits over inner tube 32 and slides along inner tube 32. The second tie 14b lies inside both main tube 16 and inside inner tube 32. Inner tube 32 and second tie 14b together act as the means for tightening first tie 14a around the patient's head. In addition, knot 24 is typically held in place by the friction between first tie 14a and inner tube 32. Alternatively, the ends of first tie 14a may be tied together to tighten knot 24. In this embodiment, knot 24 may be loosened or tightened through adjustment of first tie 14a only. There is no need to loosen or tighten second tie 14b, thereby resulting in less movement of the patient's head. FIG. 5 also shows optional pads 30 that may be added to make the securing device 10A more comfortable. As best illustrated in FIG. 8, the pad 30 may comprise an elongated tube which covers the portion of the tie 14b in contact with the patient's neck minimizing irritation to the wearer. The pads 30 are made of foam rubber, an elastomer, cotton, or another similar material, and the length and thickness of pads 30 may be varied depending on the situation. Pads 30 may be used with the first embodiment as well.

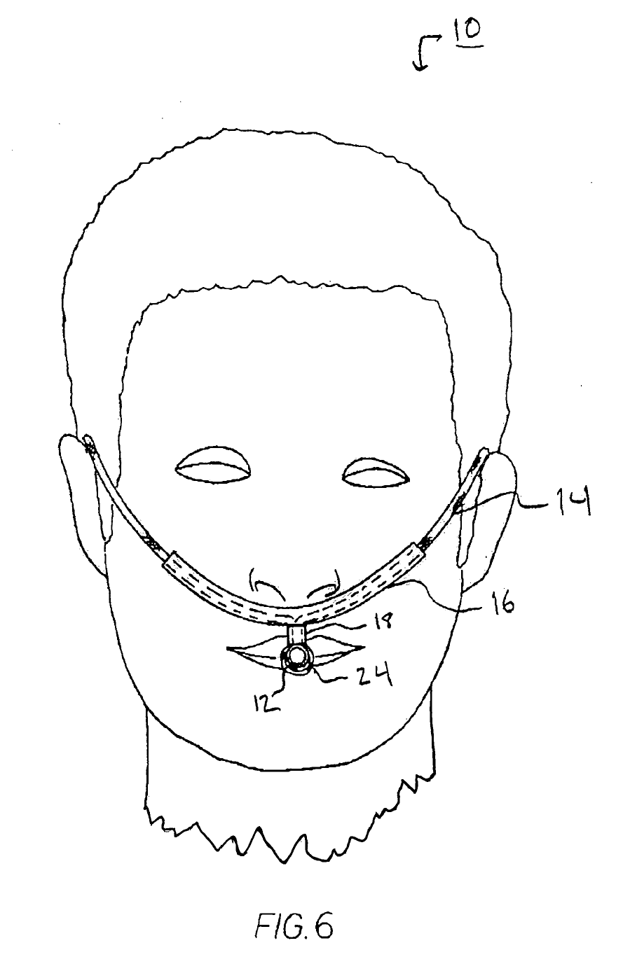

FIG. 6 shows a view of the endotracheal securing device 10 as it is fastened to a patient's face. Main tube 16 is positioned just below the patient's nose, and spacer tube 18 separates endotracheal tube 12 from main tube 16 so that endotracheal tube 12 is situated comfortably in the patient's mouth. Tie 14 wraps around the patient's head above the ears and is secured near the back or side of the head. Although not shown, the tie could also be positioned below the ears.

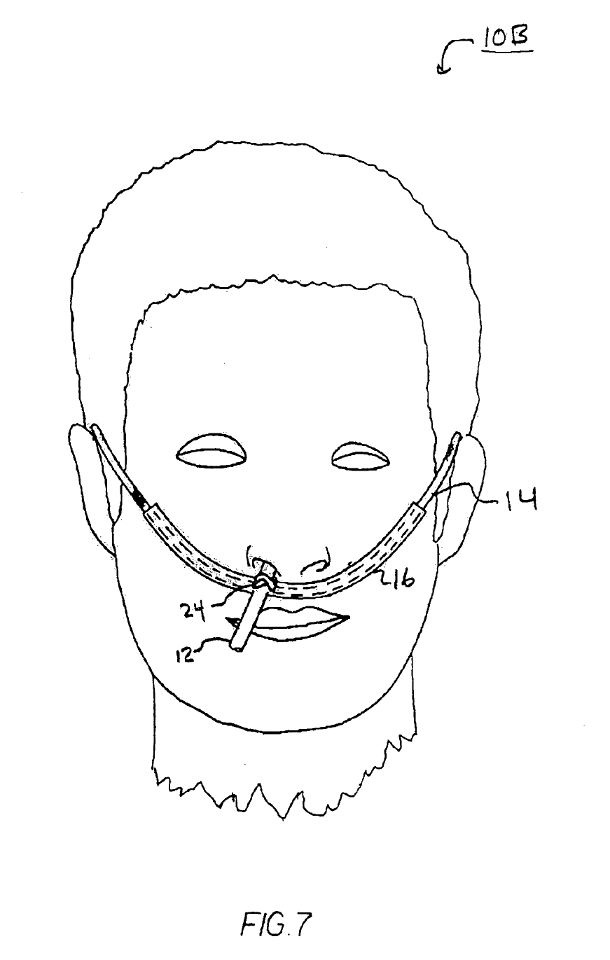

FIG. 7 shows an alternative embodiment of an endotracheal tube securing device 10B made in accordance with the present invention. This embodiment is the same as the first embodiment, except that, since the device is being used to secure a nasal endotracheal tube instead of an oral tube, the spacer tube is not required. Instead, clove hitch knot 24 protrudes directly through central orifice 16b of main tube 16. Endotracheal tube 12 is secured by knot 24, and main tube 16 is positioned just below the patient's nose so that endotracheal tube 12 is situated comfortably in the patient's nose.

While spacer tube 18 has been shown as a separate piece from main tube 16, it would be possible to form tubes 16, 18 together or to combine them into a single piece. It will be obvious to those skilled in the art that other modifications may be made to the embodiments described above without departing from the scope of the present invention.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplification presented herein above. Rather, what is intended to be covered is within the spirit and scope of the appended claims.

I claim:

1. An endotracheal tube securing device, comprising:
a main tube, including a tubular wall defining an interior, an exterior, and first and second ends;
said tubular wall also defining a central orifice extending through said tubular wall from said interior to said exterior;
a tie extending along the interior of said main tube and out said first and second ends, wherein a portion of said tie protrudes through said central orifice and forms a clove hitch knot outside of said main tube; and
means for tightening said tie around a patient's head.

2. An endotracheal tube securing device as recited in claim 1, further comprising a spacer tube surrounding said tie and positioned between said central orifice of said main tube and said clove hitch knot, wherein said tie extends from the first end of said main tube, along said interior, exits said main tube through said central orifice, passes through said spacer tube, forms a clove hitch knot beyond said spacer tube, returns through said spacer tube and through said central orifice to the interior of said main tube, and exits the second end of said main tube.

3. An endotracheal tube securing device as recited in claim 1, further comprising a removable piece of tape, wherein said tape is secured over said clove hitch knot to hold it in place, ready to receive and to be tightened around an endotracheal tube.

4. An endotracheal tube securing device as recited in claim 1, further comprising means for releasably securing said clove hitch knot so a tube can be inserted through said knot and said knot can be tightened around said tube.

5. An endotracheal tube securing device as recited in claim 4, wherein said means is a removable piece of tape, said tape being secured over said clove hitch knot to hold it in place, ready to receive and to be tightened around an endotracheal tube.

6. An endotracheal tube securing device as recited in claim 1, further comprising two soft tubular pads held in place on said tie to comfortably support said tie going as it is held against a patient's ears.

7. An endotracheal tube securing device, comprising:
a main tube, including a main tubular wall defining an interior, an exterior, and first and second ends;
said main tubular wall also defining a central orifice extending through said main tubular wall from said interior to said exterior;
a spacer tube, including a second tubular wall defining an interior and an exterior and first and second ends, said first end of said spacer tube being adjacent to said central orifice;
a first tie, said first tie extending through said first end of said main tube, along said interior of said main tube, out through said central orifice of said main tube, through said first end of said spacer tube, along the interior of said spacer tube, out through said second end of said spacer tube, forming a loop beyond said spacer tube, and then returning in through said second end of said spacer tube, along said interior of said spacer tube, out through said first end of said spacer tube, through said central orifice of said main tube, along said interior of said main tube, and out through said second end of said main tube; and
means for tightening said tie around a patient's head.

8. An endotracheal tube securing device as recited in claim 7, wherein said loop forms a knot.

9. An endotracheal tube securing device as recited in claim 8, wherein said knot is a clove hitch knot.

10. An endotracheal tube securing device as recited in claim 8, further comprising a removable piece of tape, wherein said tape is secured over said knot to hold it in place, ready to receive and to be tightened around an endotracheal tube.

11. An endotracheal tube securing device as recited in claim 9, further comprising a removable piece of tape, wherein said tape is secured over said clove hitch knot to hold it in place, ready to receive and to be tightened around an endotracheal tube.

12. An endotracheal tube securing device as recited in claim 7, wherein said means for tightening said tie around a patient's head includes a second tie extending through said main tube and out the ends of said main tube.

13. An endotracheal tube securing device as recited in claim 12, further including an inner tube extending through the interior of said main tube and containing said first tie but not containing said second tie.

14. An endotracheal tube securing device as recited in claim 13, wherein said loop forms a clove hitch knot.

15. An endotracheal tube securing device as recited in claim 14, further comprising a removable piece of tape, wherein said tape is secured over said clove hitch knot to hold it in place, ready to receive and to be tightened around an endotracheal tube.

16. An endotracheal tube securing device, comprising:
a main tube, including a tubular wall defining an interior, an exterior, and first and second ends; said tubular wall also defining a central orifice extending through said tubular wall from said interior to said exterior;
a tie extending along the interior and out the first and second ends of said main tube, wherein a portion of said tie protrudes out said central orifice and forms a knot outside of said main tube;
a removable piece of tape secured over said knot to hold it in place, ready to receive and to be tightened around an endotracheal tube; and
means for tightening said tie around a patient's head.

17. An endotracheal tube securing device as recited in claim 16, wherein said knot is a clove hitch knot.

18. An endotracheal tube securing device as recited in claim 16, further comprising a spacer tube surrounding said tie and positioned outside of said main tube, between said central orifice of said main tube and said knot.

19. An endotracheal tube securing device as recited in claim 17, further comprising a spacer tube surrounding said tie and positioned outside of said main tube, between said central orifice of said main tube and said clove hitch knot.

20. An endotracheal tube securing device as recited in claim 16, further comprising two soft tubular pads held in place on said first tie to comfortably support said tie going around a patient's ears.
